(19) United States
(12) Patent Application Publication
(10) Pub. No.: US 2010/0318176 A1
(43) Pub. Date: Dec. 16, 2010

(54) ENHANCED SYSTEMS PROCESSES AND ASSOCIATED METHODS FOR LASER TRIMMING OF GRAFTS

(76) Inventor: Donald T. Shannon, Trabuco Canyon, CA (US)

Correspondence Address: Luce, Forward, Hamilton & Scripps LLP, 2050 Main Street, Suite 600, Irvine, CA, 92614, US

(21) Appl. No.: 12/816,836

(22) Filed: Jun. 16, 2010

Related U.S. Application Data
(60) Provisional application No. 61/187,623, filed on Jun. 16, 2009.

(51) Int. Cl. A61F 2/06; A61L 33/00; B29C 63/00; B29D 23/00

(52) U.S. Cl. 623/1.13; 264/482; 427/2.25; 156/187; 156/250

(57) ABSTRACT

A novel enhanced process for laser trimming of grafts includes to heat polish the ends. Both heat polish and vapor polish are known processes in plastic processing. When PTFE is used as a graft or covering material, a problem occurs, time and temperature required will distort the ptfe material in the covering of stent graft, A simple solution is the “wetting” of the ends with and aqueous solution of FEP or PFA. A temperature lower than the melt point of PTFE would be used leaving the graft or covering in tact and unharmed. An aqueous solution of PTFE could also work provided conduction or ironing occurred.

### RELATED APPLICATION

This application claims the benefit of and full Paris Convention priority of U.S. Application Ser. No. 61/187,623, filed Jun. 16, 2009, which is hereby incorporated by reference in its entirety.

### BACKGROUND

This disclosure relates to ways to join disparate materials together in stents and/or stent-grafts, for example, enhanced ways to fuse plastics and metals involved in the same.

According to embodiments of the present disclosure, a system is disclosed, which uses heat polishing to join ends of stents.

According to embodiments of the present disclosure, a smooth transition is provided at the proximal end of a graft.

The above-mentioned features and objects of the present disclosure will become more apparent with reference to the following description.

### BRIEF DESCRIPTION OF THE DRAWINGS

FIG. 1 is a perspective view of an embodiment of a stent graft in accordance with the present disclosure;

FIG. 2 shows an embodiment of a step of a manufacturing process in accordance with the present disclosure; and

FIG. 3 shows an embodiment of a step of a manufacturing process in accordance with the present disclosure.

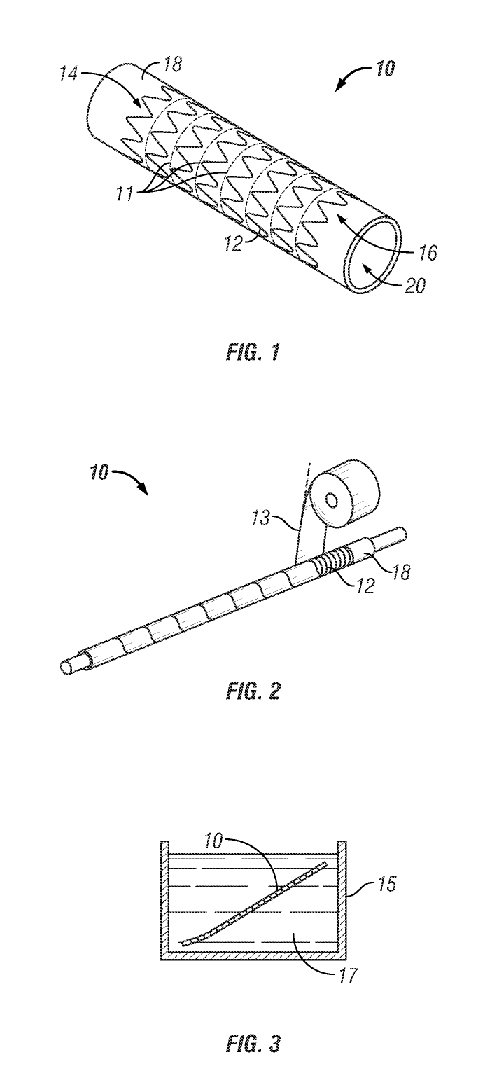

### SUMMARY OF THE DISCLOSURE

Briefly stated, a novel enhanced process for laser trimming of grafts includes to heat polish the ends. Both heat polish and vapor polish are known processes in plastic processing. When PTFE is used as a graft or covering material, a problem occurs, time and temperature required will distort the ptfe material in the covering of stent graft, A simple solution is the “wetting” of the ends with and aqueous solution of FEP or PFA. A temperature lower than the melt point of PTFE would be used leaving the graft or covering in tact and unharmed. An aqueous solution of PTFE could also work provided conduction or ironing occurred.

According to embodiments, there is a process for laser trimming of a graft, which comprises, in combination providing at least a stent or stent graft; covering said at least a stent or stent graft; trimming or cutting at least an end of said graft; heat polishing or vapor polishing at least said end.

According to embodiments, there is a process for laser trimming a stent graft, which comprises, in combination providing at least one stent and at least one covering on the at least one stent; cutting the covering at at least one end to form a cut end; treating the cut end by one or more of: heat polishing, laser trimming and laser welding; and wetting at least an end of said graft with an aqueous solution of one or more of: FEP, PFA and PTFE.

According to embodiments, there is a process for finishing a stent graft, comprising, in combination providing at least one of a graft and a stented graft; cutting at least one end of the at least one of graft and a stented graft; laser trimming the at least one end of the at least one of a graft and a stented graft; and wetting the at least one end of the at least one of a graft and a stented graft.

According to embodiments, there is a product by the processes for finishing a stent graft as described herein, whereby a resulting edge is smooth and densified and substantially non-thrombogenic.

### DETAILED DESCRIPTION

One of the problems with covered stents and stent grafts is providing the proximal end with a smooth transition.

This problem is believed to cause the failure of stent grafts in the small diameters used in the peripheral and cardiac areas.

When the covering material is cut, irregularities occur. Loose ends and cut witnesses leave rough ends which can cause micro-emboli and thrombus.

The variability in stent manufacture makes computer controlled cutting impossible without very expensive vision systems.

The easy solution would be to heat polish the ends. Both heat polish and vapor polish are known processes in plastic processing.

When PTFE is used as a graft or covering material, a problem occurs.

The time and temperature required will distort the ptfe material in the covering of stent graft.

A simple solution is the “wetting” of the ends with and aqueous solution of FEP or PFA.

A temperature lower than the melt point of PTFE would be used leaving the graft or covering intact and unharmed.

An aqueous solution of PTFE could also work provided conduction or ironing occurred and was done rapidly. A process for laser trimming of graft, which comprises, in combination, providing at least a stent or stent graft, covering said at least a stent or stent graft; trimming or cutting at least an end of said graft; heat polishing or vapor polishing at least said end.

The process further comprising “wetting” of the ends with an aqueous solution of FEP or PFA. The process of claim further comprising operating at a temperature lower than the melt point of PTFE being used leaving the graft or covering intact and unharmed.

The process of adding an aqueous solution of PTFE could also work provided conduction or ironing occurred and was done rapidly.

Embodiments of the disclosure include a process for laser trimming a stent graft, which comprises, in combination, providing at least one stent and at least one covering on the at least one stent, cutting the covering at at least one end to form a cut end, treating the cut end by one or more of heat polishing, laser trimming and laser welding, and wetting at least an end of the graft with an aqueous solution of one or more of FEP, PFA or PTFE. The cutting may be laser cutting. The covering may be seamed to take the form of a tube, or may be a tape or a film and may be helically applied or seamed to resemble a tube. The covering also may be a product of dip-coating the stent, or may be laminated by tape- or dip-coating. The tape- or dip-coating may have a lower melt temperature than the covering or a melt temperature approximately equal to that of the covering. The covering may be ironed and trimmed and may be heat polished or laser trimmed.

An exemplary stent graft 10 comprises an elongated tubular stent 12 having first and second ends 14, 16 and a sleeve or covering 18 made of graft material. The stent can be made of nitinol or any other inert, biocompatible light metal. The graft material may be ePTFE or other fluorinated polymers, ultra thin wall material, regular thin wall material, or any other inert, biocompatible material. The tubular stent graft 10 defines a passageway 20 through its interior such that blood or other biological fluids may pass through the stent graft. It should also be noted that the stent graft can flex or bend to fit in curves in blood vessels. Gaps in the stent frame enable this flexing and bending.

Exemplary methods of manufacturing a stent graft include elongating a stent frame 12 and employing laser processing using a stent cutting machine to form the desired stent design. A tubular graft member 18 is then bonded, helically applied (see FIG. 2), or otherwise secured to the stent frame 12, either to the inside or outside of the frame. Embodiments may have both an inner graft sleeve 18a and an outer graft sleeve 18b. The graft covering may be, among other things, a tube, a tape 13 or a film. In exemplary embodiments, the graft member 18 is made of ePTFE by extruding PTFE in an extruder to form a tubular extrudate. The extrudate is then expanded so it forms a fibril microstructure in which the fibrils are oriented parallel to the axis of longitudinal expansion. The resulting graft member 18 could be loaded onto a mandrel and the stent frame 12 disposed about and elongated over the graft member. Alternatively, the stent may be dip-coated to apply the covering. In exemplary embodiments, an outer graft sleeve 18b forms a bond with and inner graft sleeve 18-a. This bond can be created by a wrapping process in which tensioning is applied to the stent graft assembly. The graft covering may be laminated by a tape-coating or dip-coating process such that the tape- or dip-coating has a melt temperature lower or equal to the graft covering. Any known methods of coating stents can be used, such as those described in U.S. Patent Application Publication No. 2005/0113909, which is hereby incorporated by reference in its entirety.

Exemplary processes for finishing or trimming a stent graft will now be described. The graft material may be cut by, e.g., laser cutting performed by a spiral machine, or may be ironed or trimmed. The ends of the stent or stent-graft may then be polished to remove surface impurities or breaks in the crystal structure and provide a smooth end transition surface. Mechanical polishing techniques could be used, and exemplary embodiments use heat polishing or electropolishing because the latter techniques are more effective and don't leave scratches. Electropolishing will smooth the surface of the stent-graft as well as remove crystal lattice point impurities. Heat polishing and/or vapor polishing also may be used. Advantageously, the polishing may be done in an aqueous solution of one or more of Fluorinated Ethylene Propylene (FEP), perfluoroalkoxy (PFA) or PTFE. Aqueous PTFE could be used if conduction or ironing occurred and was done rapidly. As shown in FIG. 3, stent graft 10 may be immersed in a vessel 15 containing an aqueous solution 17 of FEP, PFA or PTFE. The use of FEP or PFA moistens the ends of the PTFE graft material to eliminate any distortion of the PTFE graft material that would otherwise occur due to the time and temperature required for certain heat polishing or vapor polishing. In exemplary embodiments, the polishing process is performed at a temperature lower than the melting point of PTFE to leave the graft or covering material intact and unharmed.

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims. Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

All references listed in the Information Disclosure Statement or other information statement filed with the application are hereby appended and hereby incorporated by reference; however, as to each of the above, to the extent that such information or statements incorporated by reference might be considered inconsistent with the patenting of this/these disclosure(s), such statements are expressly not to be considered as made by the applicant.

Thus, it is seen that systems and methods of financing entertainment projects are provided. While the methods and systems have been described in terms of what are presently considered to be the most practical and preferred embodiments, it is to be understood that the disclosure need not be limited to the disclosed embodiments, and it will be evident to one skilled in the art that various changes and modifications may be made therein without departing from the disclosure. It is intended in the appended claims to cover all such various modifications and similar arrangements that fall within the true spirit and scope of the disclosure. The scope of the claims should be accorded the broadest interpretation so as to encompass all such modifications and similar structures.

Such equivalent, broader, or even more generic terms should be considered to be encompassed in the description of each element or action. Such terms can be substituted where desired to make explicit the implicitly broad coverage to which this disclosure is entitled. It should be understood that all actions may be expressed as a means for taking that action or as an element which causes that action. Similarly, each physical element disclosed should be understood to encompass a disclosure of the action which that physical element facilitates.

It should be understood that for practical reasons and so as to avoid adding potentially hundreds of claims, the applicant has presented claims with initial dependencies only. Support should be understood to exist to the degree required under new matter laws—including, but not limited to, United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to, or actually relinquished, such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive forms so as to afford the applicant the broadest coverage legally permissible.

It should also be understood that a variety of changes may be made without departing from the essence of the disclosure. Such changes are also implicitly included in the description. They still fall within the scope of this disclosure. Further, each of the various elements of the description and claims may also be achieved in a variety of manners. This disclosure should be understood to encompass each such variation, be it a variation of an embodiment of any system embodiment, a method or process embodiment, or even merely a variation of any element of these. The present disclosure includes any and all embodiments of the following claims.

1. A process for laser trimming of a graft, which comprises, in combination:
Providing at least a stent or stent graft;
Covering said at least a stent or stent graft;
Trimming or cutting at least an end of said graft;
Heat polishing or vapor polishing at least said end.

2. Products by the process of claim 1.

3. The process of claim 1, further comprising “wetting” of the ends with an aqueous solution of FEP or PFA.

4. The process of claim 3, further comprising operating at a temperature lower than the melt point of PTFE being used leaving the graft or covering intact and unharmed.

5. The process of claim 4, further comprising providing an aqueous solution of PTFE.

6. Products by the process of claim 5.

7. A process for laser trimming a stent graft, which comprises, in combination:
providing at least one stent and at least one covering on the at least one stent;
cutting the covering at at least one end to form a cut end;
treating the cut end by one or more of: heat polishing, laser trimming and laser welding; and
wetting at least an end of said graft with an aqueous solution of one or more of: FEP, PFA and PTFE.

8. Products by the process of claim 7.

9. A process for finishing a stent graft, comprising, in combination:
providing at least one of a graft and a stented graft;
cutting at least one end of the at least one of a graft and a stented graft;
laser trimming the at least one end of the at least one of a graft and a stented graft;
and wetting the at least one end of the at least one of a graft and a stented graft

10. The process of claim 9 wherein wetting step is optional.

11. The process of claim 9 further comprising a covering which is one of: a tape and a film.

12. The process of claim 9 wherein the covering is helically applied.

13. The process of claim 9 wherein the covering is seamed to resemble a tube.

14. The process of claim 9 wherein the covering is a product of dip-coating the stent.

15. The process of claim 9 wherein the covering is laminated.

16. The process of claim 15 wherein the laminating is performed by tape- or dip-coating.

17. The process of claim 16 wherein the tape- or dip-coating has a lower melt temperature than the covering.

18. The process of claim 15 wherein the tape- or dip-coating has a melt temperature approximately equal to the covering.

19. The process of claim 18 wherein the covering is ironed and trimmed.

20. A product by the process of claim 19, whereby a resulting edge is smooth and densified and substantially non-thrombogenic.
